(19) United States
(12) Patent Application Publication
(10) Pub. No.: US 2009/0216237 A1
(43) Pub. Date: Aug. 27, 2009

(54) Instrument for Moving a Vertebra

(75) Inventors: Olivier Frezal, Rosny-sous-Bois (FR); Dominique Petit, Verton (FR); Stéphane Bette, Tiburon, CA (US)

Correspondence Address: BLANK ROME LLP, WATERGATE, 600 NEW HAMPSHIRE AVENUE, N.W., WASHINGTON, DC, 20037, US

(73) Assignee: SPINEVISION, Paris (FR)

(21) Appl. No.: 11/922,615

(22) PCT Filed: Jun. 30, 2006
(86) PCT No.: PCT/FR2006/001554

§ 371 Date: Apr. 22, 2009

(30) Foreign Application Priority Data
Jul. 1, 2005 (FR) 0552004

(51) Int. Cl. A61B 17/58

(52) U.S. Cl. 606/90

(57) ABSTRACT

The invention concerns an instrument for moving a vertebra (1) relative to another (2) comprising: a stem (3) consisting of a substantially horizontal crosspicce (5) and a first arm (4) whereof the lower end (15) has means (24) to be anchored to a first vertebra (2); and a second arm (6) whereof the lower end (10) has means (25) for being anchored to a second vertebra (1), and one upper end has a means (7) for actuating in translation said second arm (6) along a direction substantially perpendicular to the crosspiece (5), said actuating means being associated with the crosspiece (5). The second arm (6) is deformable along at least one degree of freedom between the lower end (10) of the second arm (6) and its actuating means (7).

The present invention relates to an instrument for correcting the deformation of the vertebral column and more particularly relates to an instrument for moving a vertebra relative to another.

The invention is more particularly intended for treating spondylolisthesis. Spondylolisthesis is a motion forward of a vertebra with respect to the vertebra below it. Such pathology has severe neurological consequences and causes pain and a loss of mobility of the lower limbs, in the most serious cases. Age or some congenital diseases may cause this pathology.

Techniques making it possible to treat such pathology are known. Arthrodesis exists, which consists in placing or replacing the moved vertebra using pedicle screws and longitudinal linking members, then fusing such vertebra with the adjacent one. The posterolateral fusion of the vertebra with the adjacent vertebra is performed by stripping the bone and supplying a graft. Such posterolateral arthrodesis can be completed by an intersomatic fusion with insertion of a graft and possibly an implant or intersomatic cage between the vertebral bodies.

In order to correct spondylolisthesis, it is known, in the prior art, to use instruments and implants making it possible to move vertebrae. However, such instruments or such implants impart only one motion kinematics on the vertebra. Thus, they are not easily adapted to a patient's particular anatomy. More particularly, during the motion of the vertebra, the required traction effort can be very important because of the kinematics imparted by the instrument or the implant, which does not always correspond to the path of the least effort. More particularly, it may be necessary for the vertebra which is to be moved, to tilt first, to get over the obstacle composed by the vertebral endplate of the adjacent vertebra, prior to being brought backwards afterwards. Consequently, failure risks exist during this operation and more particularly the risk of pulling out the pedicle screws or of breaking the pedicles. Such risks are all the more important since the patient is old and the quality of his bones is poor. Besides, the instruments and implants of the prior art do not give access to the disc space to make an intersomatic fusion.

The European patent application EP 0 499 037 attempts to limit such risks, by providing a vertebra realignment device, comprising two arms which can be articulated. Said arms can thus, prior to the correction operation, be positioned on the corresponding vertebrae while taking into account the patient's anatomy and the position of the moved vertebra. However, once the pre-adjustment is carried out, the arms are locked in the selected position and the vertebra is moved with a stress.

From the American patent U.S. Pat. No. 5,782,831 is also known an instrument intended to reduce the motion of a vertebra positioned between adjacent vertebrae. Said reduction device comprises an arm intended to be fixed on a longitudinal linking member mounted on pedicle screws previously fixed to vertebrae positioned on either side of the moved vertebra. Such an instrument has a few disadvantages, however. More particularly, its implementation requires the presence of at least two vertebrae, positioned on either side of the moved vertebra, so as to make it possible to fix a longitudinal linking member. Such an instrument thus does not allow the realignment of an end vertebra.

The invention aims at remedying such problems by providing an instrument for moving a vertebra relative to another with a kinematics without stress, by adapting to each patient's particular anatomy and giving a free access to the disc space, so as to make an intersomatic fusion.

For this purpose and according to a first aspect, the invention provides an instrument for moving a vertebra relative to another comprising:
a stem consisting of a substantially horizontal cross-piece and a first arm whereof the lower end has means to be anchored to a first vertebra; and
a second arm whereof the lower end has means for being anchored to a second vertebra, and one upper end has means for actuating in translation said second arm along the direction substantially perpendicular to the cross-piece, said actuating means being associated with the cross-piece,
   said instrument being characterized in that the second arm is deformable along at least one degree of freedom between the lower end of the second arm and its actuating means.

Thus, an anteroposterior force is exerted between the two vertebrae while keeping some clearance for the second vertebra relative to the first one. Consequently, the stresses on the vertebrae are reduced and the misalignment between the lower end and the actuating means of the second arm is balanced.

Besides, the so configured instrument requires the “sacrifice” of only one vertebra to realign the moved vertebra. The word “sacrifice” means the use of a well-positioned vertebra for anchoring the instrument, the straightening of the moved vertebra being then carried out relative to the vertebra carrying the instrument.

Besides, the instrument also offers a certain number of position adjustments which make it possible to adapt it to the patient's anatomy and to give the surgeon access to the inter-vertebral disc to perform the intersomatic fusion. For example, it is possible to angularly and longitudinally adjust the position of the actuating means of the second arm relative to the lower end of the first arm. It is also possible to laterally move the cross-piece relative to the lower end of the first arm.

Besides, it is also possible to carry out the correction operation and to maintain the obtained correction without the longitudinal linking member intended for the bone fixing and fusing being positioned. Such possibility is particularly interesting since it gives the surgeon access to the inter-vertebral disc for performing the intersomatic arthrodesis, at any moment of the correction operation.

When the correction and the intersomatic arthrodesis are completed, the surgeon can connect the longitudinal linking member to the pedicle screws and even apply compression/distraction maneuvers when the instrument is positioned, since the anteroposterior correction is maintained. Once the final position of the vertebra is satisfactory, the surgeon can fix it by tightening the longitudinal linking members on the pedicle screws and then the instrument can be removed for moving the vertebra.

Other objects and advantages of the invention will appear while reading the following description and referring to the appended drawings, in which:

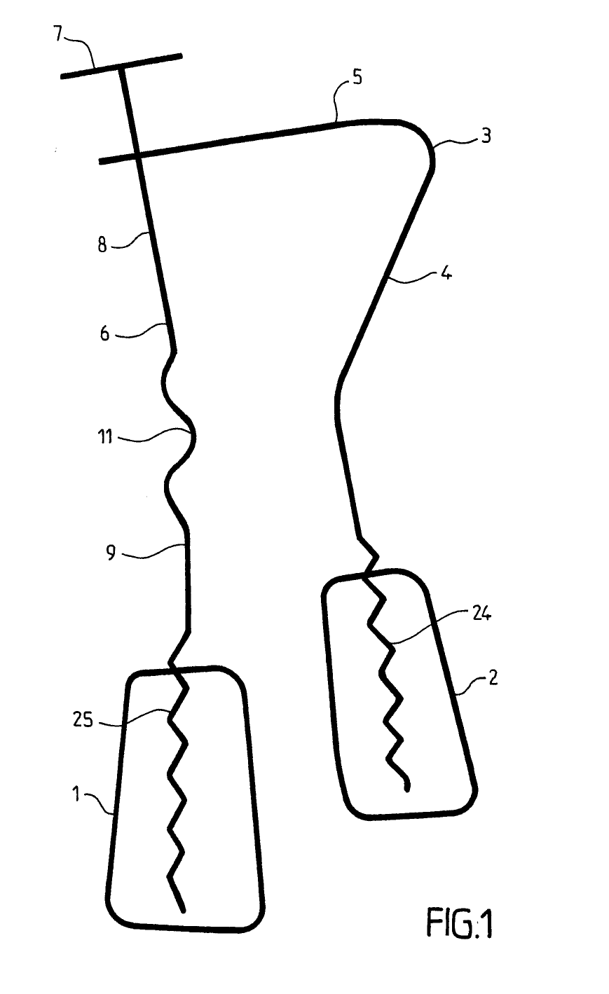

FIG. 1 shows a principle diagram of the instrument according to the invention;

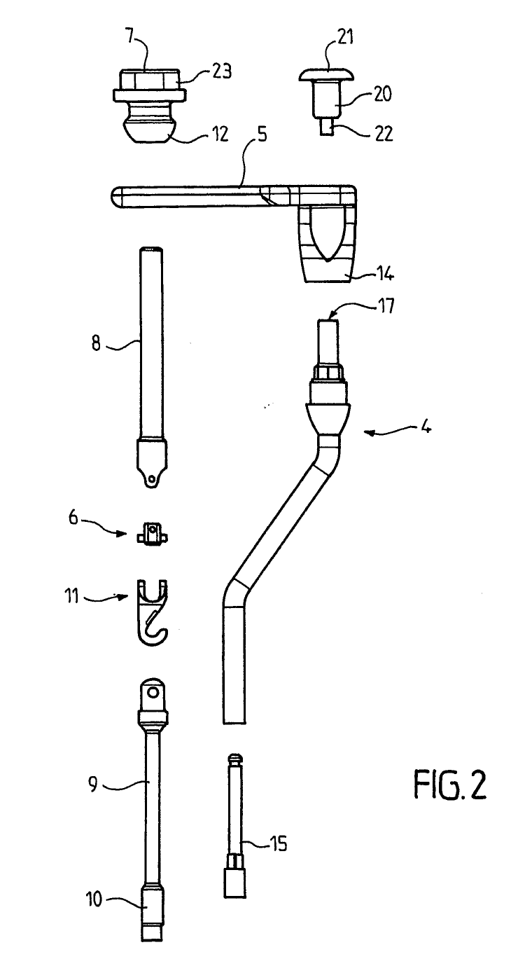

FIG. 2 shows an exploded view of the instrument according to the invention;

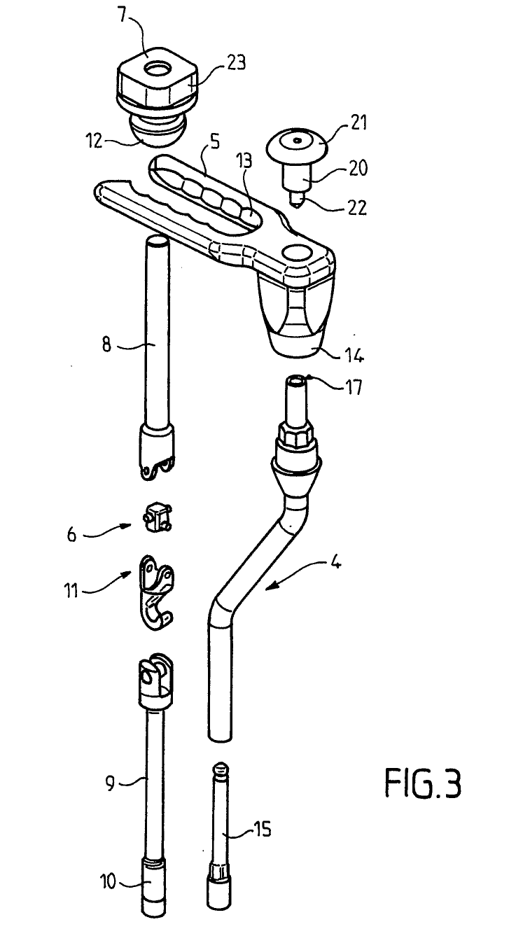

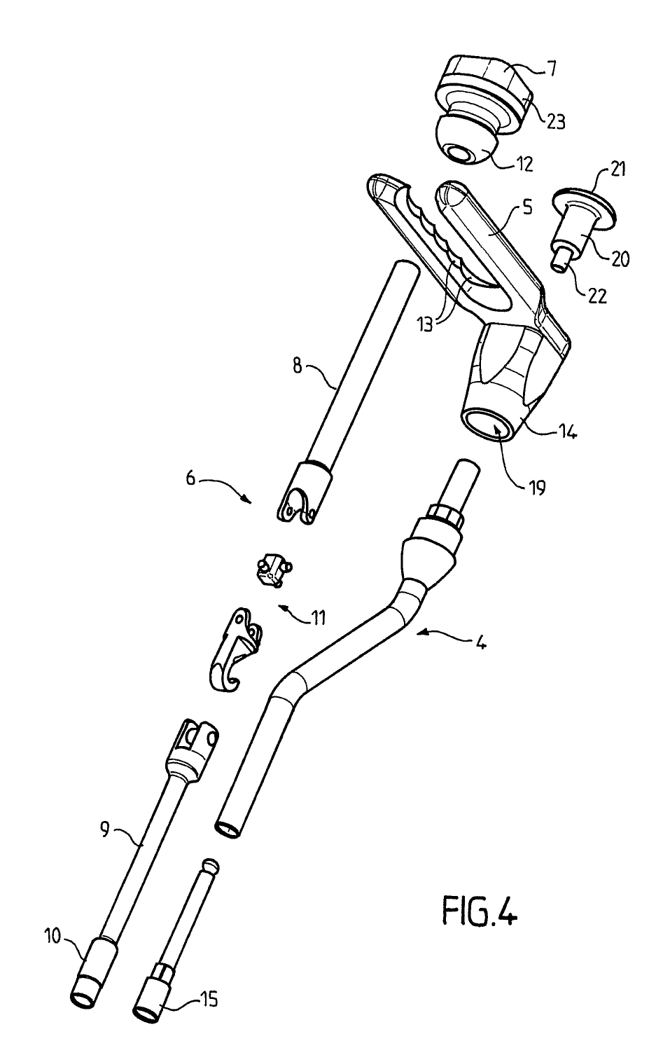

FIGS. 3 and 4 show exploded perspective views of the instrument;

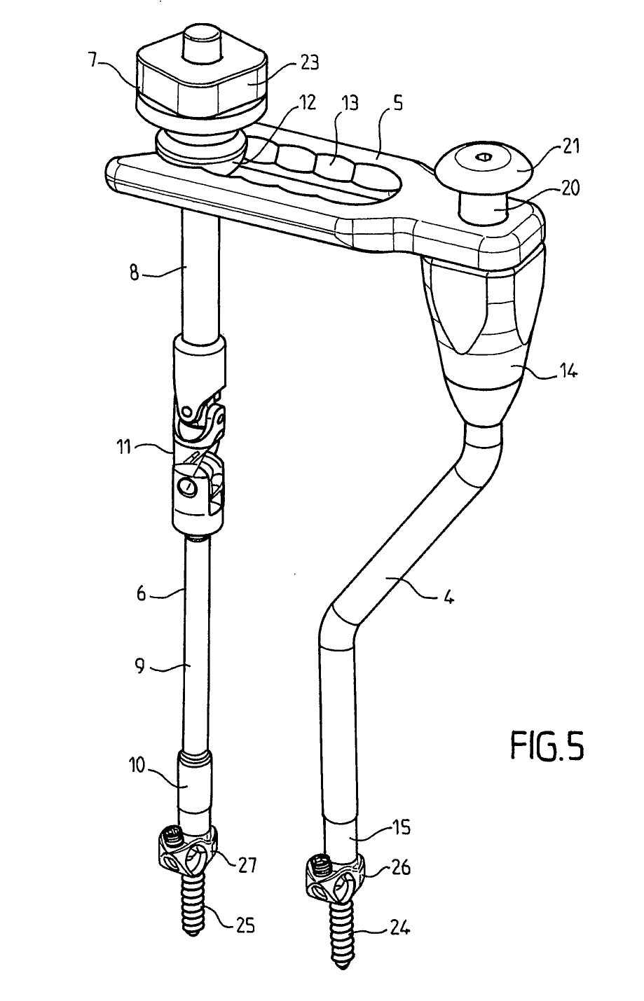

FIG. 5 shows a perspective view of the instrument according to the invention;

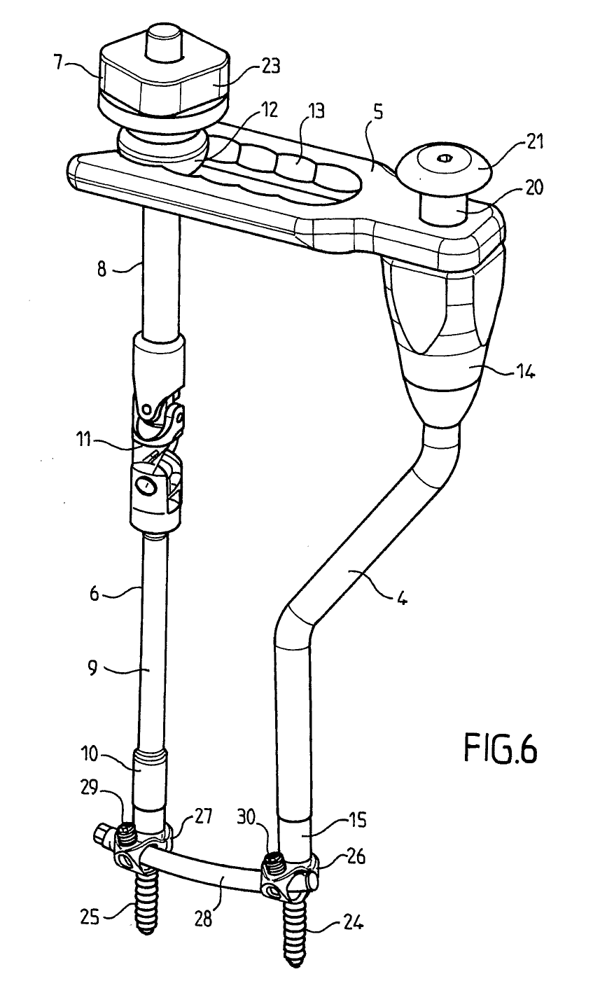

FIG. 6 shows a perspective view of the instrument, with the connectors receiving a longitudinal linking member; and

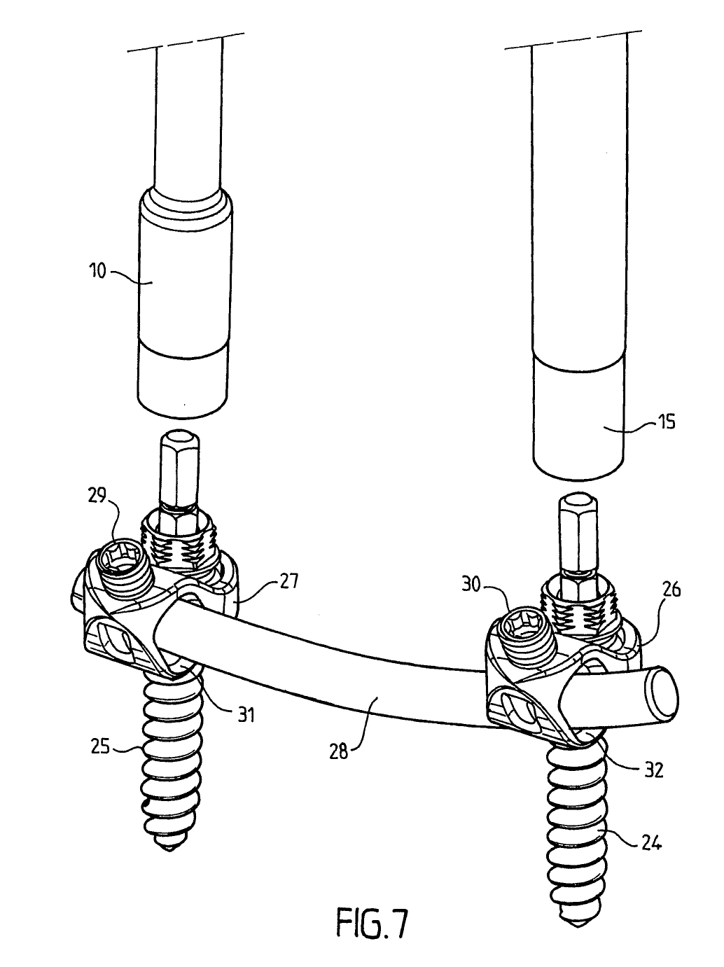

FIG. 7 shows a detailed view of the connectors receiving a longitudinal linking member.

The instrument for moving a vertebra 1 relative to another vertebra 2 such as shown in FIG. 1 consists of a stem 3. This stem 3 consists of a first vertical arm 4 and a horizontal cross-piece 5. In the embodiment shown, the stem 3 has a handle 14 making a correct gripping of the instrument possible.

The lower end 15 of the first arm 4 has means to be anchored to a first vertebra 2.

The instrument also includes a second arm 6 a lower end of which has means to be anchored to a second vertebra 1 and an upper end of which has a actuating means 7 for moving in translation said second arm 6 along a direction substantially perpendicular to the cross-piece 5. The actuating means 7 is mounted on the cross-piece 5.

The anchoring means 24, 25 of the arms 4, 6 on vertebrae 1, 2 are pedicle screws.

In one embodiment, the pedicle screws are positioned in cavities positioned on the lower ends 10, 15 of the arms.

In another embodiment of the invention, the pedicle screws are positioned in cavities for the connectors 26, 27 which can be made integral with the lower ends 10, 15 of the arms. For this purpose, the connectors have threads making it possible to make them integral with the lower ends 10, 15 of the arms.

It should be noted that the pedicle screws are also advantageously used for fixing a longitudinal linking member 28, as shown in FIGS. 6 and 7, which makes it possible to fix vertebrae together and makes the bone fusion possible. The linking member 28 is positioned upon completion of the correction operation. Advantageously, such as shown in FIGS. 6 and 7, the connectors 26, 27 have cavities 31, 32 for receiving the longitudinal linking member 28 and screws 29, 30 make it possible to lock the linking member 28 in the cavities 31, 32.

The second arm 6 is deformable along at least one degree of freedom. The degree of freedom makes it possible not to impart a motion kinematics on the second vertebra 1. Then, the motion of the second vertebra 1 is adapted to the anatomy of the patient's vertebral column, since it follows a least effort path during the translation of the second arm 6.

Besides, the misalignment between the lower end 10 of the second arm 6 and its actuating means 7 is balanced. Consequently, the actuating means 7 can be positioned with a faulty alignment relative to the end 10 of the second arm without causing mechanical stresses on the second vertebra 1 and on the screws. The position of the actuating means 7 can then be adjusted.

In a not shown embodiment of the invention, at least a portion of the second arm 6 is made of a flexible material, allowing it to be deformed. This flexible portion can be a cable, a deformable metal part or it can be made in a polymer material.

In the embodiment shown in FIGS. 2 to 4, the second arm 6 consists of two shafts 8, 9 connected by a connection member 11 having at least one degree of freedom.

Advantageously, the connection element 11 has at least two degrees of freedom.

In the embodiment shown in FIGS. 2, 3 and 4, the connection element 11 between the two shafts is a universal joint.

It should be noted, however, that the connection element used can also be a ball and socket joint or a swivel pin.

In the embodiment shown, the actuating means 7 of the second arm consist of a spherical nut 12 associated with the cross-piece 5 and a knob 23 interdependent with the spherical nut 12.

It should be noted that the word “spherical” means, in this case, an object a portion of which is inscribed in a sphere.

The second arm 6 is threaded and the knob 23 makes it possible to drive into rotation the nut 12 about the second arm 6. The rotation of the nut 12 about the second arm 6 causes the translation of the second arm 6 which thus allows the motion of the second vertebra 1.

The cross-piece 5 has the shape of a fork. At least one spherical cavity 13 making it possible to receive a nut 12 is arranged between the two prongs. The spherical cavities 13 are arranged to allow for the rotation of the nut 12 in the cavity.

Advantageously, the cross-piece 5 has several spherical cavities 13 making it possible to receive a nut 12, so as to allow for the longitudinal adjustment of the position of the actuating means 7. Thus, at the beginning of the operation, the spherical nut 12 is positioned in one of the cavities according to the selected utilization position.

Advantageously, the first arm 4 is a double bend arm. The particular shape of this arm 4 makes it possible to avoid arm interference problems which are the result of the patient's anatomy. For example, the vertebra can have a lordosis angle which requires a convergence from the pedicle screws and thus the arms 4, 6.

In the embodiment shown in FIGS. 2 to 4, the cross-piece 5 is rotatingly movable about the first arm 4. The cross-piece 5 has a cavity 19 which makes it possible to receive the upper end of the first rotatingly movable arm 4 in said cavity 19. A push button 20 has a pushing area 21 and a body engaging into the cavity 19 on the cross-piece 5. The lower end 22 of the push button 20 has a shape substantially complementary to a cavity 17 of the first arm. Thus, when the user presses the push button 20, the rotation of the cross-piece 5 about the first arm 4 is locked. Such mobility and the locking possibility make it possible to arrange for a working space, for carrying out surgery on vertebrae. Thus, it is possible to insert a graft or an intersomatic cage between the vertebrae 1, 2.

1. An instrument for moving a vertebra (1) relative to another vertebra (2) comprising:
a stem (3) consisting of a substantially horizontal cross-piece (5) and a first arm (4) whereof the lower end (15) has means (24) for anchoring to a first vertebra (2); and
a second arm (6) whereof the lower end (10) has means (25) for anchoring to a second vertebra (1), and one upper end has means (7) for actuating in translation said second arm (6) along a direction substantially perpendicular to the cross-piece (5), said actuating means being associated with the cross-piece (5),
said instrument being characterized in that the second arm (6) is deformable along at least one degree of freedom between the lower end (10) of the second arm (6) and its actuating means (7).

2. An instrument for moving a vertebra (1) according to claim 1, characterized in that the second arm (6) is consisting of two shafts (8, 9) linked by a connection member (11) having at least one degree of freedom.

3. An instrument for moving a vertebra (1) according to claim 2, characterized in that said connection member (11) is a universal joint.

4. An instrument for moving a vertebra (1) according to claim 2, characterized in that the connection member (11) is a ball and socket joint.

5. An instrument for moving a vertebra (1) according to claim 2, characterized in that said connection member (11) is a swivel pin.

6. An instrument for moving a vertebra (1) according to claim 1, characterized in that at least a portion of the second arm (6) is made of a flexible material.

7. An instrument for moving a vertebra (1) according to claim 6, characterized in that at least a portion of the second arm (6) is a cable.

8. An instrument for moving a vertebra (1) according to claim 6, characterized in that at least one portion of the second arm (6) is made of a polymer material.

9. An instrument for moving a vertebra (1) according to claim 6, characterized in that at least a portion of the second arm (6) is a deformable metal part.

10. An instrument for moving a vertebra (1) according to one of claims 1 to 9, characterized in that the actuating means (7) of the second arm (6) consist of a nut (12) associated with the cross-piece (5), the second arm having a thread and the rotation of the nut about the second arm driving the translation of the second arm (6).

11. An instrument for moving a vertebra (1) according to claim 10, characterized in that the nut is spherical.

12. An instrument for moving a vertebra (1) according to one of claims 10 or 11, characterized in that the actuating means (7) further comprises a knob (23) integral with the spherical nut (12) and making it possible to drive said nut (12) in rotation about the second arm (6).

13. An instrument for moving a vertebra (1) according to one of claims 11 or 12, characterized in that the cross-piece (5) has a shape of a two-pronged fork, at least one spherical cavity (13) making it possible to receive the spherical nut (12) being arranged between such two prongs, said spherical nut (12) being rotatingly movable in this spherical cavity (13).

14. An instrument for moving a vertebra (1) according to claim 13, characterized in that the cross-piece (5) has several spherical cavities (13) making it possible to receive the nut (12), so as to allow the longitudinal adjustment of the position of the actuating means (7).

15. An instrument for moving a vertebra (1) according to any one of claims 1 to 14 characterized in that said stem (3) has a handle (14).

16. An instrument for moving a vertebra (1) according to any one of claims 1 to 15, characterized in that the first arm (4) is a double bend arm.

17. An instrument for moving a vertebra (1) according to any one of claims 1 to 16, characterized in that the cross-piece (5) is rotatingly movable about the first arm (4).

18. An instrument for moving a vertebra (1) according to claim 17, characterized in that the cross-piece (5) has a cavity (19) arranged for receiving a push-button (20), said push-button (20) making it possible to lock the rotation of the cross-piece (5) about the first arm (4).

19. An instrument for moving a vertebra (1) according to any one of the preceding claims, characterized in that the means for anchoring (24, 25) the arms (4, 6) on the vertebrae (1, 2) are pedicle screws.

20. An instrument for moving a vertebra (1) according to claim 19, characterized in that the pedicle screws are located in cavities positioned at the lower ends (10, 15) of the arms.

21. An instrument for moving a vertebra (1) according to claim 19, characterized in that the pedicle screws are positioned in cavities for connectors which can be interdependent with the lower ends (10, 15) of the arms.

22. An instrument for moving a vertebra (1) according to one of claims 19 to 21, characterized in that the pedicle screws can also be used for fixing a longitudinal link member (28).
